(19) United States
(12) Patent Application Publication
(10) Pub. No.: US 2006/0095050 A1
(43) Pub. Date: May 4, 2006

(54) Large diameter sheath

(75) Inventors: David Ernest Hartley, Subiaco (AU); David A. Drewes JR., Bloomington, IN (US); Fred T. Parker, Unionville, IN (US); Jay A. Dittman, Indianapolis, IN (US)

Correspondence Address: COOK GROUP PATENT OFFICE, P.O. BOX 2269, BLOOMINGTON, IN, 47402, US

(73) Assignees: William A. Cook Australia Pty. Ltd., Brisbane (AU); Cook Incorporated, Bloomington, IN (US)

(21) Appl. No.: 11/226,611

(22) Filed: Sep. 14, 2005

Related U.S. Application Data
(60) Provisional application No. 60/609,767, filed on Sep. 14, 2004.

(51) Int. Cl. A61F 11/00

(52) U.S. Cl. 606/108; 604/527; 264/171.2

(57) ABSTRACT

A large diameter, flexible, kink-resistant and rotatable introducer sheath (10) for percutaneous delivery of a contained and implantable medical device in the vasculature of a patient. The introducer sheath includes a reinforcement such as a flat wire coil (23) fitted about an inner, lubricous material such as polytetrafluoroethylene tube (22). A wire braid (25) is placed around the coil to give good transfer of rotational forces. An outer tube (27) of a heat formable polyamide material is heat formed and compressed through the spaces between the wires of the braid and turns of the wire coil to mechanically connect the outer tube to the roughened outer surface of the inner tube. The durometer of the outer tube can be varied to effect the flexibility of the sheath. A radiopaque marker (42) is positioned at the distal end of the coil and around the inner tube for radiographic visualization.

### CROSS-REFERENCE TO RELATED APPLICATIONS

This application claims priority of provisional application Ser. No. 60/609,767, filed Sep. 14, 2004.

### TECHNICAL FIELD

This invention relates generally to a medical device and, in particular, to a delivery catheter or sheath and, more particularly, to a flexible, kink-resistant delivery catheter or sheath.

### BACKGROUND OF THE INVENTION

Introducer sheaths are well known for laparoscopic and percutaneous vascular access and typically comprise polytetrafluoroethylene or fluorinated ethylene propylene. These sheaths are of a thin-wall construction, but tend to kink. Increasing the thickness of the sheath wall minimally improves the level of kink resistance, which is still unacceptable.

Sheaths used in hemofiltration and dialysis, in particular, are prone to kinking since they remain positioned in a patient's body for a long time. While positioned in a patient, the sheath may be bent or pinched off and, as a result, kink due to repeated use or patient movement. A kinked sheath is unusable and cannot be straightened while positioned in the body of a patient. Consequently, the sheath must be removed, leaving an enlarged, bleeding opening, which typically cannot be reused. Vascular access is then attempted at an alternative site, and the procedure is restarted. Restarting the procedure causes a time delay, which may be life threatening. In some cases, an alternative site is not available for introducing another sheath.

Another problem with thin-wall sheaths is that an emergency room physician will typically kink an introducer sheath while inserting various catheters therethrough during emergency procedures. Small diameter introducer sheaths are also typically bent and kinked under the time constraints of an emergency situation. As a result, a new sheath must be introduced at the same or another access site.

Introducer sheaths are described in U.S. Pat. Nos. 4,634,432; 4,657,772; and 4,705,511. These introducer sheaths utilize a helical coil spring and a cylindrical wall formed by dipping the spring in a protective coating composition, which completely surrounds the spring. The coating composition comprises a thermoplastic polymer material dissolved in a solvent solution. Although this introducer sheath appears to be more kink-resistant and flexible than a polytetrafluoroethylene sheath, the cylindrical wall is approximately twice as thick as that of the polytetrafluoroethylene sheath with the same inside diameter. The increased outside diameter of this introducer sheath significantly increases the size of the access site, which further accentuates the problem of bleeding.

Although introducer sheaths are typically utilized for initial access to, for example, the vascular system, a flexible, kink-resistant delivery catheter or sheath is also desirable for delivering implantable medical devices to a deployment site well within the vascular system of a patient. Although delivery catheters or sheaths are known for delivering implantable medical devices, large diameter catheters or sheaths are susceptible to kinking, particularly where the implantable medical device or pusher does not have a uniform diameter to reinforce the delivery catheter or sheath along its entire length. As a result, these large diameter delivery catheters or sheaths are susceptible to kinking particularly when the physician exerts any forward pressure or force to push the delivery catheter or sheath through an area of thrombus or calcification in the vascular system or through tortuous vessels.

A further problem exists that during delivery of such implantable devices it may be necessary to rotate the introducer sheath to correctly position an implantable device with respect to vasculature. The use of sheaths including coils as discussed above has been proposed but the coil does not allow rotation to be transmitted as the coil can allow the sheath to wind up or unwind on itself. Hence it is a further object to provide a large diameter sheath with an improved ability to transmit rotational torque.

It is desired to provide a large diameter catheter or sheath that is less prone to kinking than existing catheters or sheaths. It is further desired to provide such a device that may be readily tracked as it is manipulated through the vascular system.

The term large diameter is intended to mean a sheath having an inner tube having a passageway extending longitudinally therethrough with a diameter of from about 14 to 36 French.

Throughout this specification the term distal with respect to the deployment device or a portion of the deployment device is that part that is further away from the physician or user when the device is being used and the term proximal means the part of the deployment device or a portion of the deployment device that is closer to the physician or user when the device is being used. Hence the proximal end would be that end that the physician holds and the distal end would be that end that is in a patient.

### SUMMARY OF THE INVENTION

Hence the present invention is said to reside in an introducer sheath comprising an inner tube having a passageway extending longitudinally therethrough, said passageway having a substantially uniform diameter of from 14 to 36 French, a coil having a plurality of coil turns and a wire braid extending longitudinally around said inner tube and an outer tube positioned around said wire braid, the coil and the inner tube; and the outer tube being connected to inner tube through the wire braid and between the coil turns.

The wire braid may be inside or outside of the coil.

Alternatively the invention is said to reside in a introducer sheath comprising an inner tube having a passageway extending longitudinally therethrough, said passageway having a substantially uniform diameter of from 14 to 36 French; a coil having a plurality of coil turns extending longitudinally around said inner tube, a wire braid around the coil; and an outer tube positioned around said wire braid, the coil and the inner tube and the outer tube being connected to inner tube through the wire braid and between the coil turns.

A predetermined uniform spacing between the coils may be also utilized since extremely wide spacing weakens the wall and creates a rough surface. Narrow spacing may not allow sufficient room for connecting the outer tube to the inner tube. In the preferred embodiment, the coil comprises a flat wire coil for further improving the strength of the introducer sheath.

The wall of the inner tube advantageously prevents the coil turns from extending into the inner tube passageway. As a result, the inner tube passageway has a uniform diameter for passing the largest possible diameter catheter therethrough. In contrast, the protrusion of coil turns into the passageway establishes a varying diameter, which limits the size of the catheter passable therethrough. The inner tube preferably comprises a lubricous material such as polytetrafluoroethylene, which presents a slippery surface for easy insertion of a catheter therethrough. Furthermore, the inner tube includes a smooth inner surface for resisting the formation of blood clots thereon. The inner tube also advantageously includes a rough outer surface for improving the connection of the outer tube thereto through the uniform spacing of the coil turns.

Preferably the coil has a predetermined spacing of between about 0.004 and 0.08 inch (0.1 and 2 mm), and each coil turn of said coil has a width between about 0.005 and 0.030 inch (0.012 and 0.76 mm) and a thickness of from 0.003 to 0.007 inch (0.007 mm to 0.017 mm).

The braided wire is preferably formed from stainless steel wire and has a wire diameter of from 0.001 to 0.006 in (0.025 mm to 0.15 mm).

Preferably the braid has a pic in the range of 20 to 100. More preferably, the pic would be in the 50 to 80 range. ‘Pic’ is a measure of the number of cross over points per inch of length of the braid.

In a preferred embodiment the braid is a 16 wire braid (8 wires going one direction, 8 going the other) in a 2 over 2 configuration.

The outer tube advantageously comprises a heat formable polyamide material such as nylon for mechanically connecting with the rough outer surface of the inner tube. The sheath further comprises a heat shrinkable tube positioned around the outer tube for compressing the outer tube between the uniform spacing of the compression-fitted coil turns and mechanically connecting the outer tube to the rough surface of the inner tube when heated. The heat formable polyamide material is also advantageously self-leveling for providing a smooth outer surface which also reduces the formation of blood clots thereon.

Preferably the distal ends of the inner and outer tubes extend beyond the distal end of the coil. The distal end of the outer tube is preferably tapered and extends beyond the distal end of the inner tube to advantageously prevent the inner tube from presenting a rough edge or surface, which may cause injury to the vessel wall. The inner diameter of the passageway about the distal ends of the inner and outer tubes is uniform to again minimize the formation of blood clots on the inner surface of the inner tube.

Preferably the proximal ends of the inner and outer tubes also extend beyond the proximal end of the coil and are flared for attachment to a connector.

In another aspect of the present invention, a coil having an inner diameter smaller than the outer diameter of the inner tube is wound and compression fitted around the inner tube. This advantageously eliminates the necessity to collapse the inner tube for insertion into the passage of the flat wire coil. This also advantageously eliminates the formation of any wrinkles in the inner tube when the collapsed inner tube is expanded to form a compression fit against the flat wire coil.

A radiopaque marker may be positioned adjacent the distal end of the coil to improve visualization of the sheath when inserted in a patient.

In a further form, the invention is said to reside in a sheath for an endovascular deployment device, the sheath comprising; an inner tube of a plastics material; a coil of a flat wire wound onto the inner tube; a wire braid over the coil; and a thermoplastic outer later.

The invention, in an alternative form, comprises a method of manufacturing an introducer sheath, comprising the steps of:
positioning an inner tube over a mandrel, said inner tube having a substantially uniform internal diameter of from about 14 to 36 French;
positioning a coil over said inner tube, said coil having a plurality of turns, said turns having substantially uniform spacings therebetween;
positioning a wire braid over said coil;
positioning a polymeric outer tube over said inner tube, coil and wire braid to comprise a sheath assembly;
positioning a heat shrink tube over the sheath assembly;
heating said heat shrink tube and said sheath assembly so that a portion of said outer tube melts and flows between the wires of the wire braid and said coil turns to bond with said inner tube; and removing said mandrel and said heat shrink tube from said sheath.

To further advantageously control the flexibility of the delivery catheter or sheath, the second or outer tube may have a durometer having a range from 20 through 85 on the Shore D hardness scale. The second or outer tube comprises a compound of first and second polymers, preferably polyamide or more preferably nylon materials to vary the durometer and flexibility of the delivery catheter or sheath. In one preferred embodiment, the first polymer has a durometer of approximately 40 on the Shore D hardness scale and, the second polymer material has a durometer of approximately 63 on the Shore D hardness scale. To enhance radiographic visualization of the delivery catheter or sheath, the compound of polymer materials for the outer tube also includes a radiopaque material ranging from 35 through 85 percent by weight and preferably 40 percent by weight of a high density radiopaque material such as tungsten.

To also advantageously control the flexibility of the large diameter delivery catheter or sheath, each of the uniform width spacings of the preferred flat wire coil may range from between about 0.004 and 0.08 inch (0.1 and 2 mm) inches and more preferably from 0.007 through 0.009 inch (0.017 mm to 0.023 mm). To further advantageously control the flexibility and kink-resistance of the delivery catheter and sheath, each turn of the coil has a width ranging from 0.05 inches through 0.030 inches (0.012 and 0.76 mm) and preferably a width of 0.017 inches (0.43 mm). In addition, the coil has a thickness ranging from 0.003 inches through 0.007 inches (0.007 mm to 0.017 mm) and has a preferred thickness of 0.005 inches (0.013 mm).

To further advantageously radiographically differentiate the distal end of the delivery catheter or sheath, a radiopaque marker tube is disposed adjacent the distal end of the reinforcement or coil and around the inner tube. This radiographic marker tube includes a polyamide material and a high density radiopaque material being higher in density than the radiopaque material of the outer tube and preferably being greater than 40 percent by weight. In addition, a side port is advantageously positioned at the distal end of the reinforcement or flat wire coil and through the inner and outer tube as well as the radiopaque marker tube to inject or infuse contrast media into the vascular system for radiographic visualization of the delivery catheter or sheath and contained medical device.

The large diameter delivery catheters or sheaths of the present invention provide for the delivery of large diameter contained devices for delivery to, for example, the aorta and iliac arteries. Such implantable medical devices include stents and stent-graft devices for the repair or exclusion of aneurysms.

### BRIEF DESCRIPTION OF THE DRAWING

This then generally describes the invention but to assist with understanding reference will now be made to the accompanying drawings which show preferred embodiments of the invention.

In the drawings:

FIG. 1 depicts an introducer or delivery device incorporating a sheath according to one embodiment of the present invention;

FIGS. 2A to 2F depicts the various components to be put together to form an introducer sheath according to one embodiment of the present invention;

FIG. 3 depicts a partially sectioned view of an introducer sheath formed from the components of FIG. 2; and

FIGS. 4A and 4B show a partially sectioned view and a side view of another embodiment of an introducer sheath according to the present invention.

### DETAILED DESCRIPTION

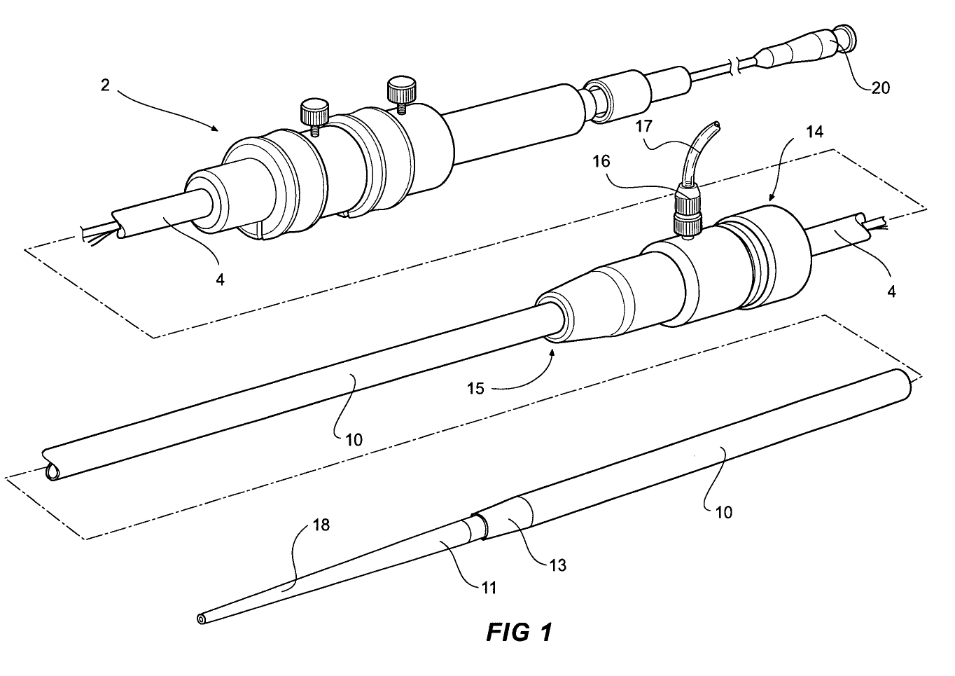

FIG. 1 depicts a delivery device 2 incorporating a flexible, kink-resistant and rotatable introducer sheath 10 according to one embodiment of the invention. The delivery device 2 has a delivery catheter 4 which extends to a distal tapered nose cone dilator 11 longitudinally through the passageway of the sheath 10. The introducer sheath extends from a tapered distal end 13 which includes a radiopaque marker to a connector valve and manipulator 14 attached about proximal end 15 of the sheath. Connector valve 14 includes a silicone disk (not shown) for preventing the backflow of fluids therethrough. The disk includes a slit for the insertion of nose cone dilator 11 and delivery catheter 4. The stent graft or implantable device (not shown) is carried on the delivery catheter 4. Connector 14 also includes side arm 16 to which polyvinyl tube 17 is connected for introducing and aspirating fluids therethrough. Nose cone dilator 11 includes tapered distal end 18 for accessing and dilating a vascular access site over a well-known and commercially available wire guide. The guide is inserted in the vessel with an introducer needle using, for example, the well-known percutaneous vascular access Seldinger technique. A well-known male Luer lock connector hub 20 is attached at the proximal end of the delivery catheter 4 for connection to syringes and other medical apparatus.

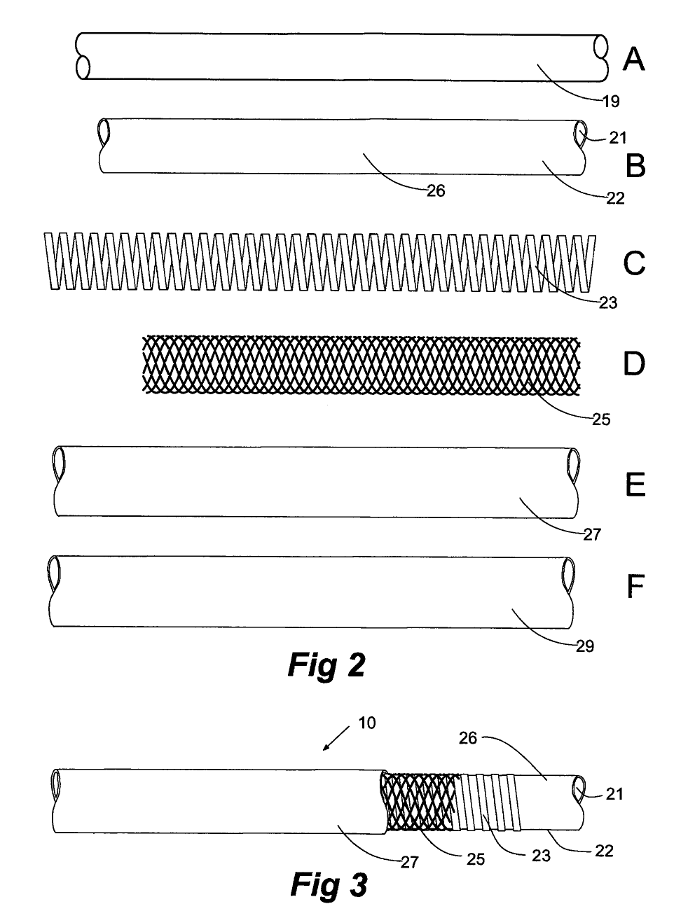

FIG. 2 shows the various components and stages used to manufacture a sheath according to one embodiment of the present invention.

The inner tube 22 is placed over a mandrel 19. The mandrel 19 is the intended size of the passageway through the finished sheath. Preferably the outer surface 26 of the inner tube is roughened to facilitate adhesion of other components as will be discussed later.

Inner tube 22 is formed from a lubricous material tube such as polytetrafluoroethylene. The lubricous polytetrafluoroethylene material presents a slippery inner surface 21 for the easy insertion and withdrawal of the dilator as well as other catheters and medical apparatus. Inner surface 21 is also smooth and nonporous for minimizing the formation of blood clots and other thrombi thereon. Outer surface 26 of the inner tube is chemically etched in a well-known manner for forming a rough outer surface to which outer tube 27 is mechanically connected using a well-known heat shrinking and formation process. The uniform inner diameter of inner tube 22 extends the entire length of the inner tube for passing the largest possible diameter catheter therethrough.

Around the inner tube is a coil 23 which comprises a plurality of flat wire turns with uniform spacing including equal width spaces therebetween. Coil 23 may be formed from 0.003″ thick by 0.012″ wide flat rectangular stainless steel wire wound with a uniform space in the range of 0.004 to 0.08 inch between the turns of the coil. Wire coil 23 is compression fitted around the outer surface of inner tube 22 approximately 15 mm from the distal end thereof and approximately 25 mm from the proximal end thereof to maintain the uniform spacing between the turns of the coil. The coil is compression fitted by collapsing inner tube 22 and inserting the wire coil thereover. Inner tube 22 is then compressed-air expanded to engage and compression fit the inner surface of the flat wire coil. The mandrel 19 is then inserted through the passageway of the inner tube to further compresses the inner tube against the coil turns during the manufacture of the sheath as hereinafter described. The coil preferably is positioned away from the distal and proximal ends of the inner tube to permit tapering and flaring of the sheath. The wall of the inner tube prevents the turns of compression-fitted coil 23 from protruding into inner tube passageway 21.

Around the wire coil a wire braid 25 is formed. The wire braid is formed from wire strands plaited tightly around the wire coil. While being tightly plaited, spaces are left to allow melt flow through the wires of the braid as discussed below. A wire braid for this application may be a 16 wire braid (8 wires going one direction, 8 going the other) in a 2 over 2 configuration (each wire goes over 2 wires, then under 2, then over 2, etc., instead of over and under each wire). Other configurations and numbers of wires may also be useful.

The braids may be in the range of 20 to 100 pics per inch (pic equals the number of wire cross over points per inch of braid). In a high pic braid, the wires form a helix wherein the angle between the axis of the wires and the axis of the braid is fairly high. In the case of a 50 pic braid, the angle is about 75 degrees. In the case of a 25 pic braid, the angle is about 45 degrees. The high pic braids are more flexible than the lower pic braids but the low pic braids transmit more torque. The pic count can be adjusted to suit the needs of the device and procedure. For the present application, a braid with a pic in the range of 20 to 100 is preferred. More preferably, the pic would be in the 50 to 80 range.

The braid material itself is typically fine stainless steel wire in the 0.001 to 0.006 in diameter range and with a high tensile strength in the range of 250K psi to 350K psi tensile. The wire can be round or some other cross sectional shape such as a flat ribbon. A flat ribbon wire has the advantage that the thickness of the braid can be minimised while maintaining a reasonable cross sectional area in the wires. A typical ribbon wire for a braid would be 0.0015 in thick by 0.005 in wide. The braid could also be made of materials other than stainless steel. Kevlar, fibreglass and carbon fibres would all be suitable braid materials.

Another braid material could be nylon monofilaments, although somewhat less effective than higher tensile materials.

As the wire braid has strands of wire extending in helically both directions around the finished sheath it is able to transmit rotation or torque applied to the sheath. In contradistinction, while the metal coil by itself provides excellent kink resistance it is not able to successfully transmit rotation or torque.

Outer tube 27 is a heat formable polyamide material such as nylon that is heat shrunk over coil 23 and braid 25, which in turn is compression fitted over inner tube 22. The outer tube is heated and compressed through the spaces between the wires of the braid and the coil turns with a heat shrink tube 29 for mechanically connecting to material of the outer tube rough outer surface of the inner tube 22. The heat shrink tube is preferably of a fluorinated ethylene propylene heat formable material. The nylon outer tube has a processing temperature range for the heat formation thereof in the range of 356 to 500 degrees Fahrenheit. After the outer tube is heat shrunk onto the roughened surface of the inner tube, the shrink tube is removed therefrom, and a taper formed at the distal end of the sheath.

Depicted in FIG. 3 is a partially sectioned view of introducer sheath 10 according to the present invention. The sheath comprises inner tube 22, flat wire coil 23 compression fitted there around, the wire braid 25 around the coil and outer tube 27 mechanically connected to the roughened outer surface of the inner tube through the spacings of the coil and the braid.

An external taper can be formed at the distal end of the outer tube to assist with deployment during use. To enable this taper to be formed the coil and the braid may be terminated shortly before the distal end of the sheath. Similarly the coil and the braid may be terminated shortly before the proximal end of the sheath to enable the proximal end to be flared to enable it to be mounted into the connector valve and manipulator 14 attached about proximal end 15 of the sheath 10 (see FIG. 1).

A radiopaque marker sleeve may be positioned distally from the flat wire coil over the inner tube near the distal end of the sheath. The radiopaque marker can comprise an elastomer, such as nylon. Preferably, the marker has a formulation similar to or the same as that of outer tube 31 to enhance thermal bonding during the heat shrink process. Also, it is preferred that the durometer of the elastomer is similar to or the same as that of the outer tube. In this manner, the flexibility of the sheath is not adversely affected by the presence of the marker. The percent filler of radiopaque material in the marker may vary depending on the loading capacity of the particular elastomer used. For a nylon marker having a durometer of about 40, the radiopaque loading may be between about 40 and 90 wt %, preferably about 80 wt %, of the total weight of the radiopaque marker.

Preferably, the radiopaque material is a material commonly used for such purposes, such as tungsten. However the radiopaque material may comprise any well-known radiopaque filler material that is compatible with the matrix of the radiopaque marker, and that may be loaded in the matrix at sufficiently high loading levels to enable an operator to distinguish the marker from the remainder of the sheath under fluoroscopy. Alternatively the radiopaque marker comprises a filler with, for example, 10 percent iridium with the remainder being a platinum material.

In an alternative embodiment the flat wire coil may be wound around the inner tube to form the compression fit between the inner tube and wire coil. The coil is wound around the inner tube by expanding and wrapping the coil around the inner tube using, for example, a commercially available lathe and a transfer mechanism attached to the carriage of the lathe. This winding technique improves the manufacturing process and maintains closer tolerances for the uniform spacing between the turns of the coil. In addition, there is no need to compress or collapse the inner tube for insertion into the passage of the flat wire coil. This advantageously eliminates any wrinkles in the inner tube wall and maintains closer manufacturing tolerances.

Although the preferred spacing between the reinforcement for flat wire turns of the coil are approximately 0.13 millimeters, it is also contemplated that the spacing between the reinforcement or flat wire coil turns can be varied along the length of the delivery catheter or sheath to provide variable kink resistance and/or flexibility along the entire length of the catheter or sheath. Similarly, it is also contemplated that the durometer of the second or outer tube can also be varied in combination with the reinforcement spacing to provide variable flexibility along the catheter or sheath depending on the medical use, application or procedure being performed.

It may be noted that when the sheath 10 bends, the outer tube 27 formed from a polyamide material such as nylon has to stretch on the outside of the bend and compress on the inside of the bend. The spacing of the coil 23 influences the ability of the sheath to stretch and compress when bent. In other words, if the coil spacing is very tight, the sheath will be very stiff because the material is not free to stretch and compress. On the other hand, if the spacing is very large, the sheath will be very flexible because the polymer can stretch and compress as required. Taken to extremes, the sheath would be too stiff to be usable if the spacing is very small and the sheath would have the tendency to kink if the spacing is too great. Varying the spacing within the useful limits of spacing will give the sheath different bending properties and can be adjusted to suit the needs of the procedure.

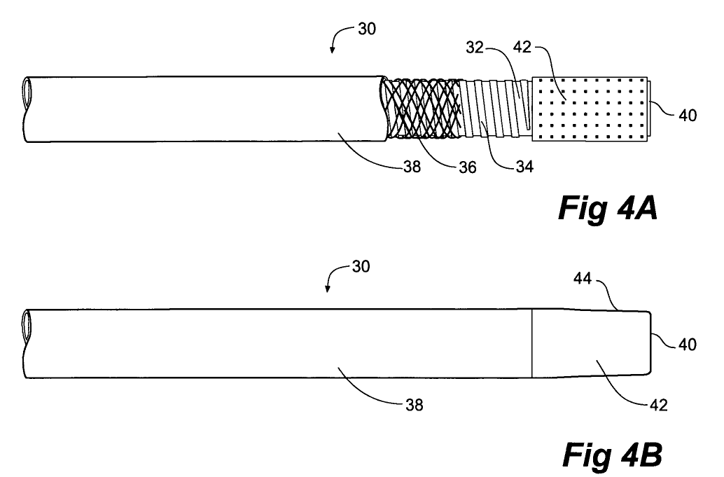

FIGS. 4A and 4B show a partially sectioned view and a side view of another embodiment of introducer sheath according to the present invention. The sheath 30 is shown partially sectioned in FIG. 4A and comprises an inner tube 32, flat wire coil 34 compression fitted around the inner tube, a wire braid 36 around the coil and outer tube 38 mechanically connected to the roughened outer surface of the inner tube through the spacings of the coil and the braid. The coil 34 terminates before the end 40 of the inner tube 32 and a radiographic marker sleeve 42 is fitted around the inner tube. The radiographic marker sleeve 42 is substantially the same material as that of the outer tube but is filled with a radiopaque filler material that is compatible with the matrix of the radiopaque marker and that may be loaded in the matrix at sufficiently high loading levels to enable an operator to distinguish the marker from the remainder of the sheath under fluoroscopy.

As shown in FIG. 4B the outer tube 38 extends to the radiographic marker sleeve 42 and then the radiographic marker sleeve 42 is joined to the outer tube 38 by welding and is tapered at 44 towards the end 40 of the sheath 30 and has a slightly rounded tip.

It is to be understood that the above-described flexible, kink-resistant and torquable introducer sheath is merely an illustrative embodiment of the principles of this invention and that other introducer sheaths may be devised by those skilled in the art without departing from the spirit and scope of this invention. It is contemplated that various other materials may be utilized for the inner, outer, and heat shrink tubes.

Throughout this specification various indications have been given as to the scope of this invention but the invention is not limited to any one of these but may reside in two or more of these combined together. The examples are given for illustration only and not for limitation.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

What is claimed is:

1. An introducer sheath, comprising:
an inner tube having a passageway extending longitudinally therethrough, said passageway having a substantially uniform diameter of from 14 to 36 French;
a coil comprising a plurality of coil turns extending longitudinally around said inner tube and a predetermined spacing between the coil turns,
a wire braid at least one of inside and outside of the coil; an outer tube positioned around said wire braid, the coil and the inner tube; and
the outer tube being connected to the inner tube through the wire braid and between the coil turns.

2. The introducer sheath of claim 1 wherein the coil comprises stainless steel.

3. The introducer sheath of claim 1 wherein said inner tube comprises a lubricous material.

4. The introducer sheath of claim 3 wherein said lubricous material comprises polytetrafluoroethylene.

5. The introducer sheath of claim 1 wherein said inner tube includes a smooth inner surface and a rough outer surface.

6. The introducer sheath of claim 1 wherein said outer tube comprises polyamide.

7. The introducer sheath of claim 1 wherein said outer tube has a durometer hardness having a range from 20 to 85 on the Shore D hardness scale.

8. The introducer sheath of claim 1 further comprising a polymeric radiographic marker tube disposed adjacent a distal end of said coil.

9. The introducer sheath of claim 8 wherein said radiographic marker tube comprises polyamide and is disposed along said sheath between said inner tube and said outer tube, said marker tube comprising a high density radiopaque material ranging between about 40 and 90 weight percent of the total weight of the marker tube.

10. The introducer sheath of claim 10 wherein said marker tube comprises nylon, and said radiopaque material is selected from the group comprising at least one of tungsten, iridium and platinum.

11. The introducer sheath of claim 1, wherein said inner tube comprises PTFE, said coil comprises stainless steel flat wire, the wire braid comprises stainless steel and said outer tube comprises nylon.

12. The introducer sheath of claim 1 wherein the wire braid comprises stainless steel wire and has a wire diameter of from 0.001 to 0.006 inches (0.025 to 0.15 mm).

13. The introducer sheath of claim 1 wherein the braid has a pic in the range of 20 to 100.

14. The introducer sheath of claim 1 wherein the braid is a 16 wire braid (8 wires going one direction, 8 going the other) in a 2 over 2 configuration.

15. The introducer sheath of claim 1 wherein the predetermined spacing is between about 0.004 and 0.08 inches (0.1 and 2 mm), and each coil turn of said coil has a width between about 0.005 and 0.030 inches (0.012 and 0.76 mm) and a thickness of from 0.003 to 0.007 inches (0.007 to 0.017 mm).

16. The introducer sheath of claim 1 wherein a proximal end of said coil is spaced from a proximal end of said sheath by about 0.5 to 5.0 inches (1.27 to 12.7 cm), and a distal end of said coil is spaced from a distal end of said sheath by about 0.1 to 2 inches (0.25 to 5.1 cm).

17. The introducer sheath of claim 1, wherein the coil comprises stainless steel flat wire.

18. The introducer sheath of claim 1, wherein the wire braid comprises a material selected from the group comprising at least one of stainless steel, Kevlar, fiberglass, nylon monofilaments and carbon fibers.

19. The introducer sheath of claim 1 wherein the coil comprises a stainless steel flat wire, a proximal end of the coil is spaced from a proximal end of said sheath by about 0.5 to 5.0 inches (1.27 to 12.7 cm), a distal end of the coil is spaced from a distal end of said sheath by about 0.1 to 2 inches (0.25 to 5.1 cm), the predetermined spacing is between 0.004 and 0.08 inches (0.1 and 2 mm), and each coil turn of the coil has a width between about 0.005 and 0.030 inches (0.012 and 0.76 mm) and a thickness of from 0.003 to 0.007 inches (0.007 to 0.017 mm); wherein the inner tube comprises a lubricous material comprising polytetrafluoroethylene and includes a smooth inner surface and a rough outer surface; wherein said outer tube comprises a polyamide and has a durometer hardness having a range from 20 to 85 on the shore D hardness scale; wherein the wire braid comprises a material selected from the group comprising at least one of stainless steel, Kevlar, fiberglass, nylon monofilaments and carbon fibers and has a wire diameter of from 0.001 to 0.006 inches (0.025 to 0.15 mm) and a pic in the range of 20 to 100; wherein the wire brain comprises a 16 wire braid (8 wires going one direction, 8 going the other) in a 2 over 2 configuration; wherein the sheath further comprises a polymeric radiographic marker tube disposed along adjacent a distal end of the coil; wherein the marker tube comprises polyamide and is disposed along said sheath between the inner tube and the outer tube, and wherein the marker tube further comprises a high density radiopaque material ranging between about 40 and 90 weight percent of the total weight of the marker tube.

20. A method of manufacturing an introducer sheath comprising the steps of:
positioning an inner tube over a mandrel, said inner tube having a substantially uniform internal diameter of from about 14 to 36 French;
positioning a coil over said inner tube, said coil having a plurality of turns, said turns having substantially uniform spacings therebetween;
positioning a wire braid over said coil;
positioning a polymeric outer tube over said inner tube, coil and wire braid to comprise a sheath assembly;
positioning a heat shrink tube over the sheath assembly;
heating said heat shrink tube and said sheath assembly so that a portion of said outer tube melts and flows between the wires of the wire braid and said coil turns to bond with said inner tube; and
removing said mandrel and said heat shrink tube from said sheath.
